(19) United States
(12) Patent Application Publication
(10) Pub. No.: US 2017/0189219 A1
(43) Pub. Date: Jul. 6, 2017

(54) Lordosis Adjustable Back Brace Device

(71) Applicant: Wade K. Jensen, Dakota Dunes, SD (US)

(72) Inventor: Wade K. Jensen, Dakota Dunes, SD (US)

(21) Appl. No.: 15/464,306

(22) Filed: Mar. 20, 2017

Related U.S. Application Data
(63) Continuation-in-part of application No. 14/453,077, filed on Aug. 6, 2014.
Application No. 14/453,077 is a continuation-in-part of application No. 13/707,262, filed on Dec. 6, 2012, now Pat. No. 9,237,956.
(60) Provisional application No. 62/310,455, filed on Mar. 18, 2016.
(60) Provisional application No. 62/325,967, filed on Apr. 21, 2016.

(51) Int. Cl. A61F 5/02

(52) U.S. Cl. CPC A61F 5/028

(57) ABSTRACT

A lordosis adjustable back brace device supports a desired shape of the spine. The device includes a belt for extending around a torso of a human. The belt has a front closure and is positionable in a closed loop. A support panel is coupled to the belt such that the support panel projects from an inner surface of the closed loop towards a center of the closed loop. The support panel abuts the torso and has a curved surface facing the center of the closed loop. A curvature of the curved surface extending between lateral edges of the belt is adjustable.

### (b) CROSS-REFERENCE TO RELATED APPLICATIONS

This application claims benefit of the provisional application 62/310,455 filed Mar. 18, 2016, and provisional application 62/325,967 filed Apr. 21, 2016. This application is further a continuation in part of pending non-provisional application Ser. No. 14/453,077 filed on Aug. 6, 2014, which is a continuation in part of issued U.S. Pat. No. 9,237,956.

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

(1) Field of the Invention

(2) Description of Related Art Including Information Disclosed Under 37 CFR 1.97 and 1.98

The disclosure and prior art relates to back brace devices and more particularly pertains to a new back brace device for adjusting curvature of the device for supporting a desired shape of the spine.

### (h) BRIEF SUMMARY OF THE INVENTION

An embodiment of the disclosure meets the needs presented above by generally comprising a belt for extending around a torso of a human. The belt has a front closure and is positionable in a closed loop. A support panel is coupled to the belt such that the support panel projects from an inner surface of the closed loop towards a center of the closed loop. The support panel abuts the torso and has a curved surface facing the center of the closed loop. A curvature of the curved surface extending between lateral edges of the belt is adjustable.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top rear side perspective view of a lordosis adjustable back brace device according to an embodiment of the disclosure.

FIG. 2 is a top rear side perspective view of a two support panel embodiment of the disclosure.

FIG. 3 is a top rear side perspective view of a two support panel independent adjustment embodiment of the disclosure.

FIG. 4 is a top rear side perspective view of a single support panel on a backing panel embodiment of the disclosure.

FIG. 5 is a front view of interchangeable backing panels of an embodiment of the disclosure.

FIG. 6 is a top view of an embodiment of the disclosure.

FIG. 7 is a side view of an embodiment of the disclosure in use.

FIG. 8 is a front view of a top sliding embodiment of the disclosure.

FIG. 9 is a front view of a bottom sliding embodiment of the disclosure.

FIG. 10 is a front view of a top and bottom sliding embodiment of the disclosure.

FIG. 11 is a side view of an alternative sliding embodiment of the disclosure.

FIG. 12 is a front side perspective view of a single adjustment two support panel on a backing panel embodiment of the disclosure.

FIG. 13 is a front side perspective view of an embodiment of the disclosure.

FIG. 14 is a detailed view of an embodiment of the disclosure.

FIG. 15 is a side view of an embodiment of the disclosure.

FIG. 16 is a front side perspective view of a support panel of an embodiment of the disclosure.

FIG. 17 is a rear view of a lacing configuration for a single pull embodiment of the disclosure.

FIG. 18 is a lacing configuration for a double pull embodiment of the disclosure.

FIG. 19 is a front view of a single adjustment two support panel on a backing panel embodiment of the disclosure.

FIG. 20 is a side view demonstrating pelvic incidence.

FIG. 21 is a side view demonstrating spinal alignment relative to a C7 plumb line.

FIG. 22 is a chart of total lumbar lordosis and distribution of curvature in degrees determined from pelvic incidence.

FIG. 23 is a detailed partial side view of a support panel of an embodiment of the disclosure.

FIG. 24 is a front view of an embodiment of the disclosure.

### (j) DETAILED DESCRIPTION OF THE INVENTION

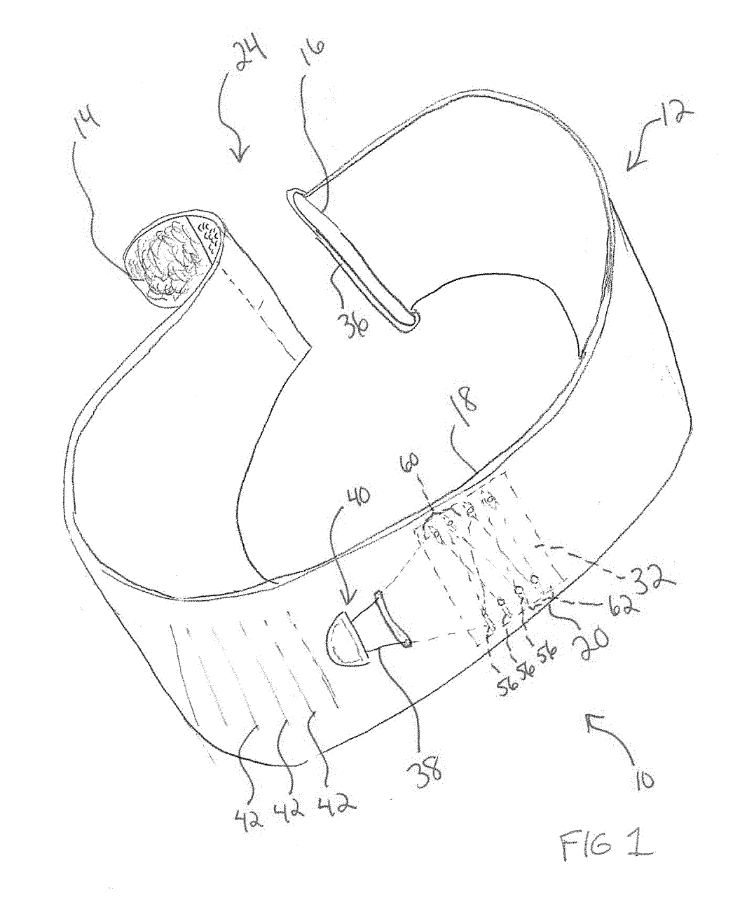

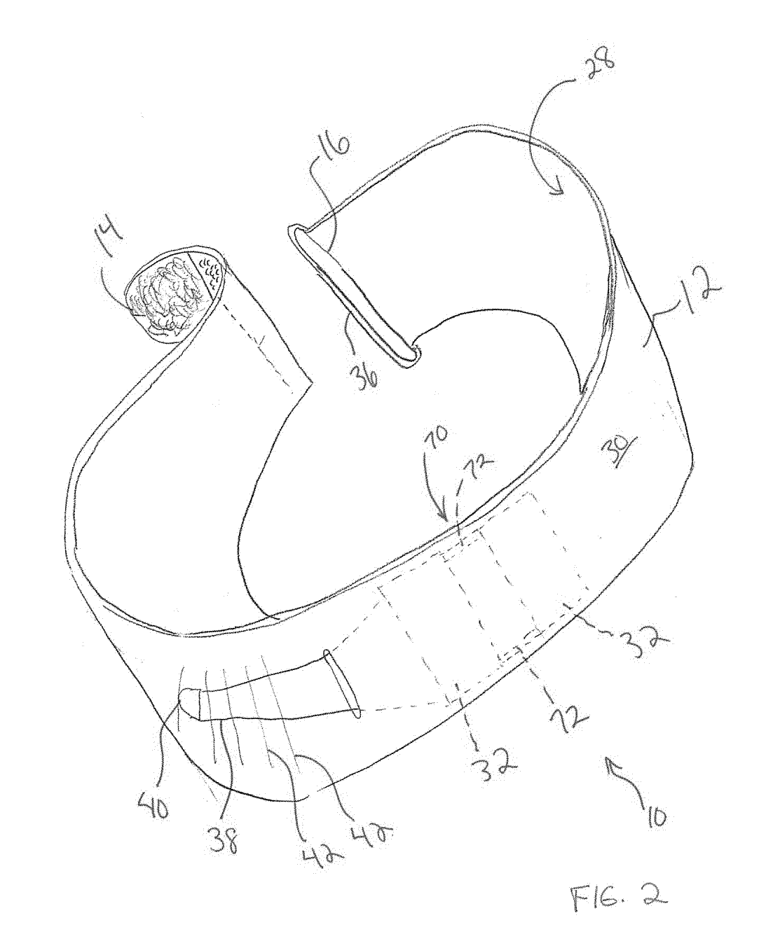

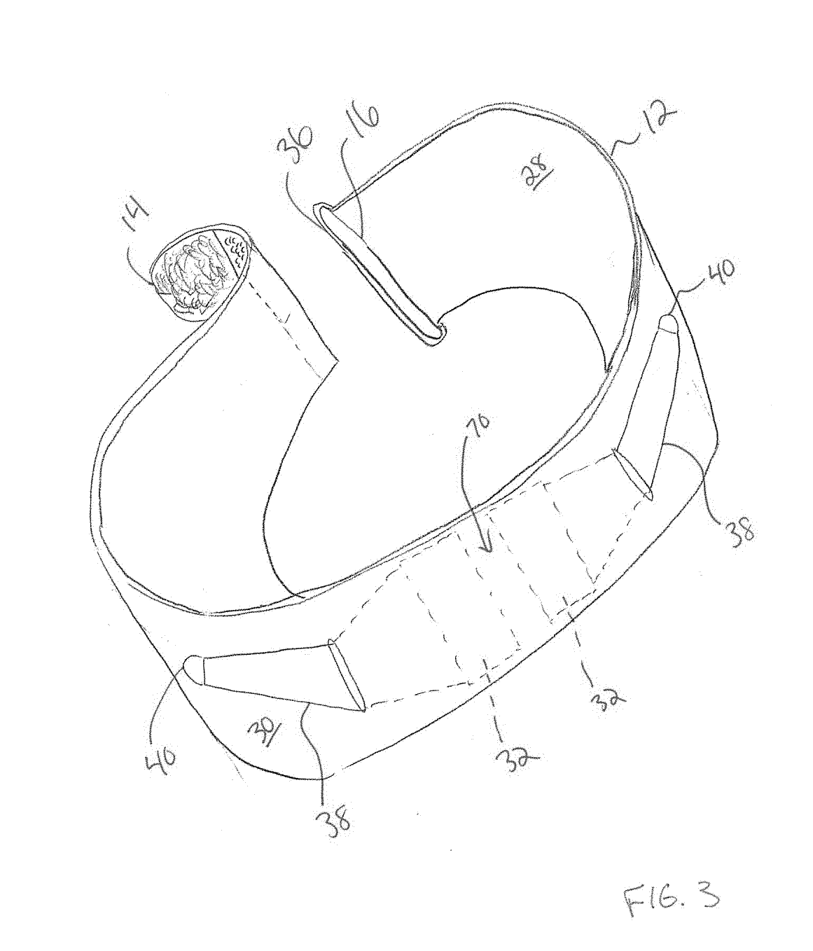

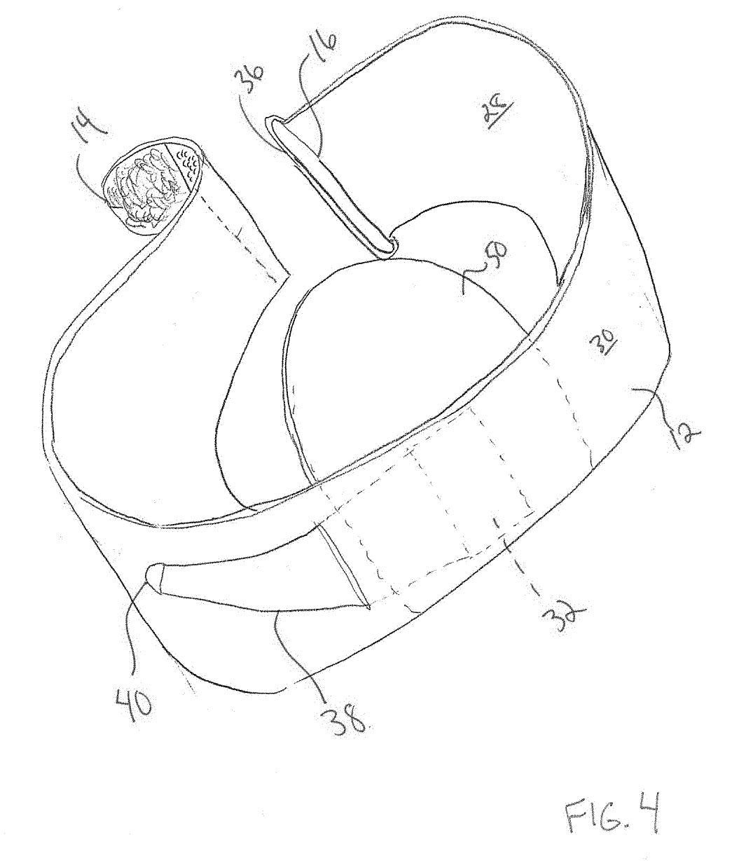

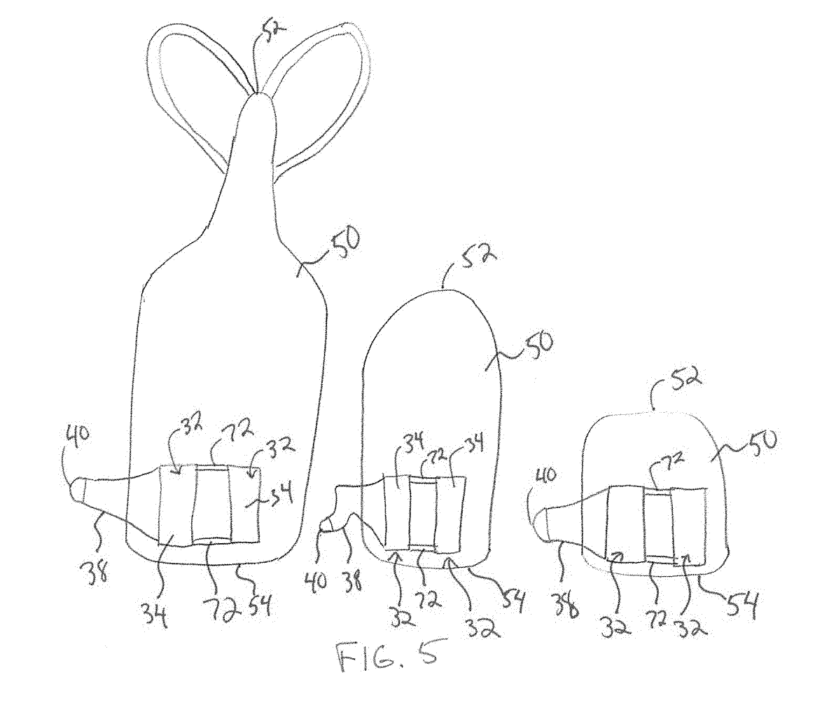

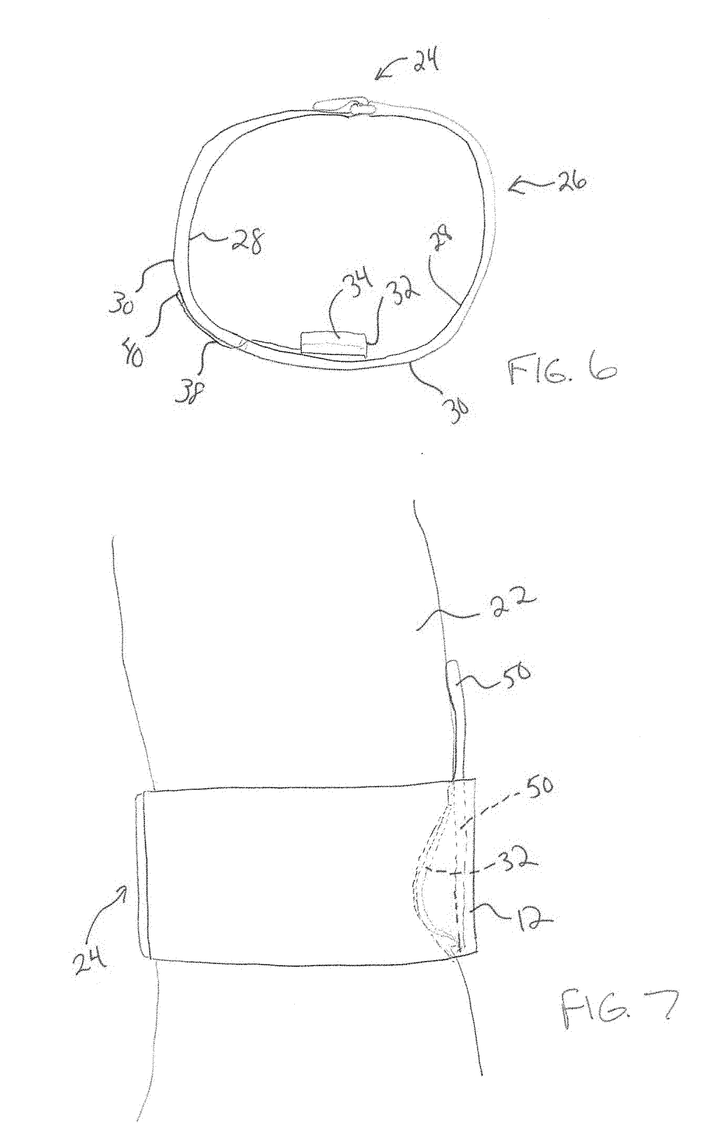

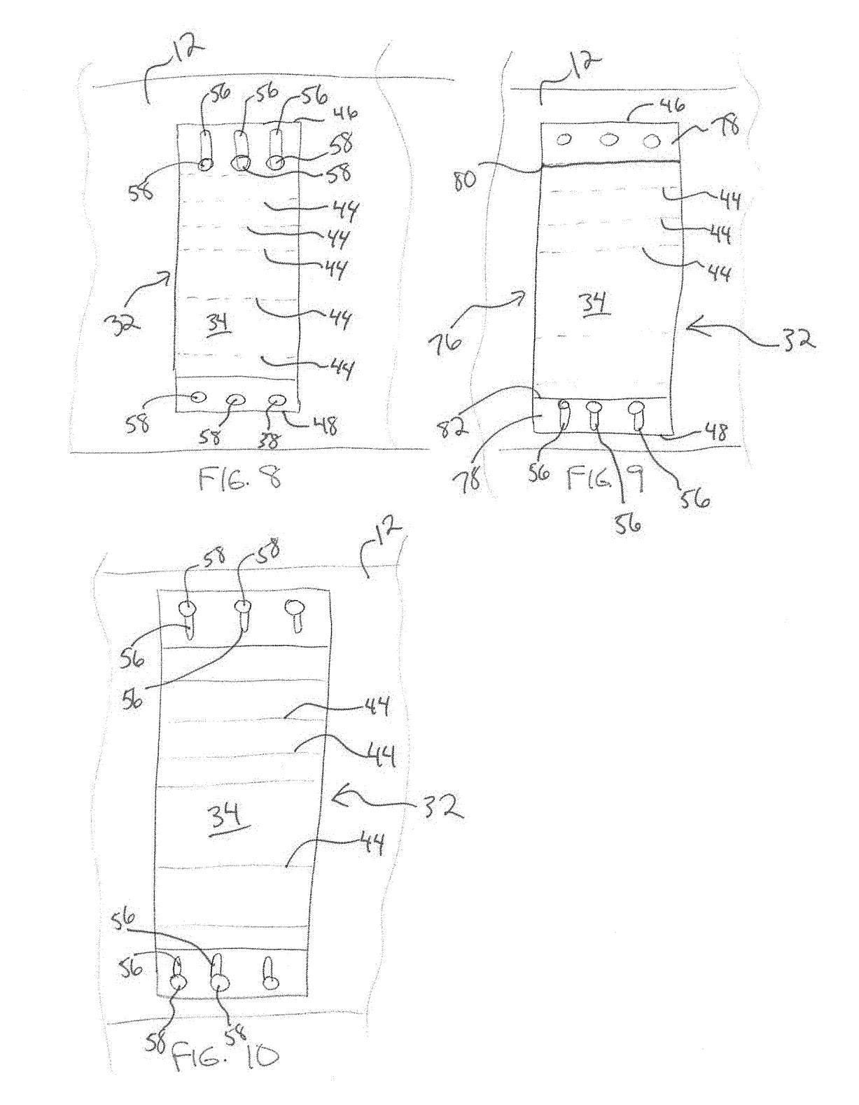

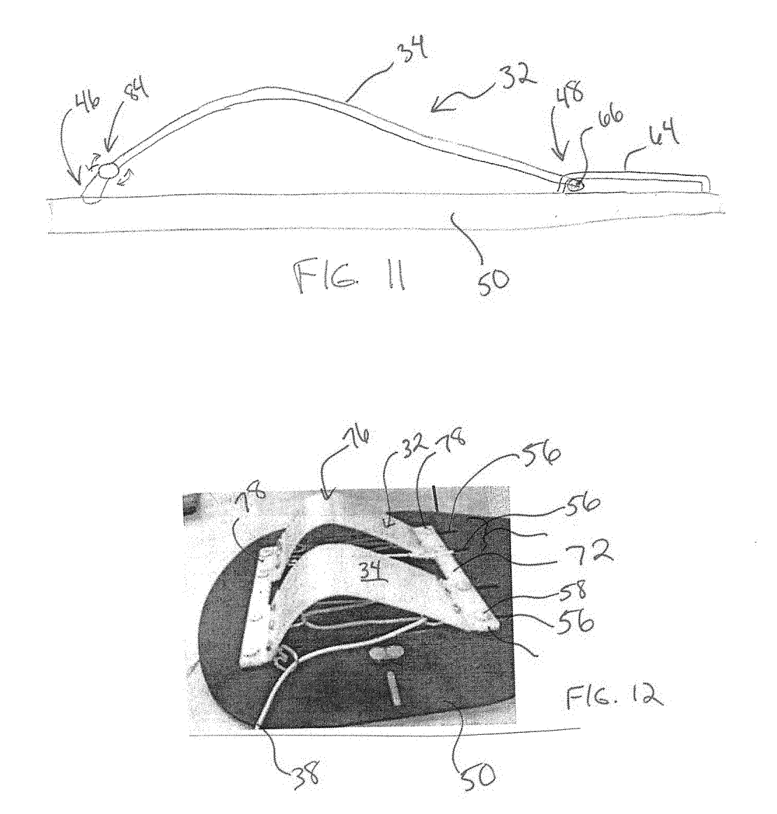

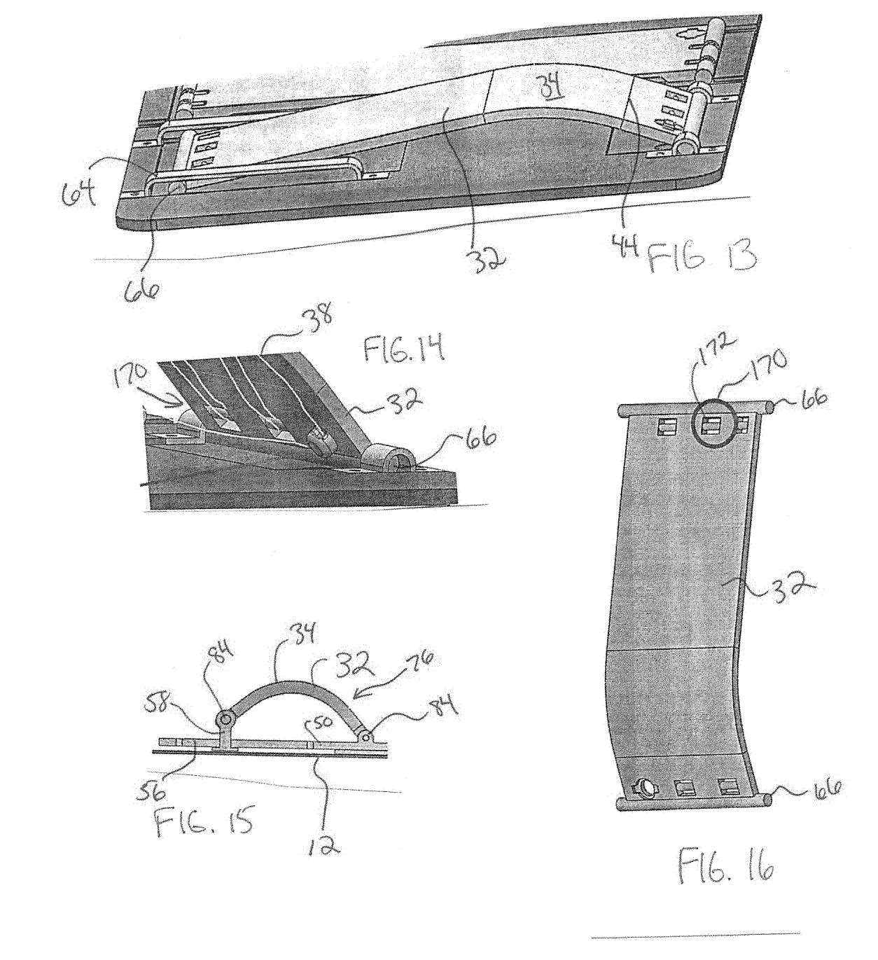

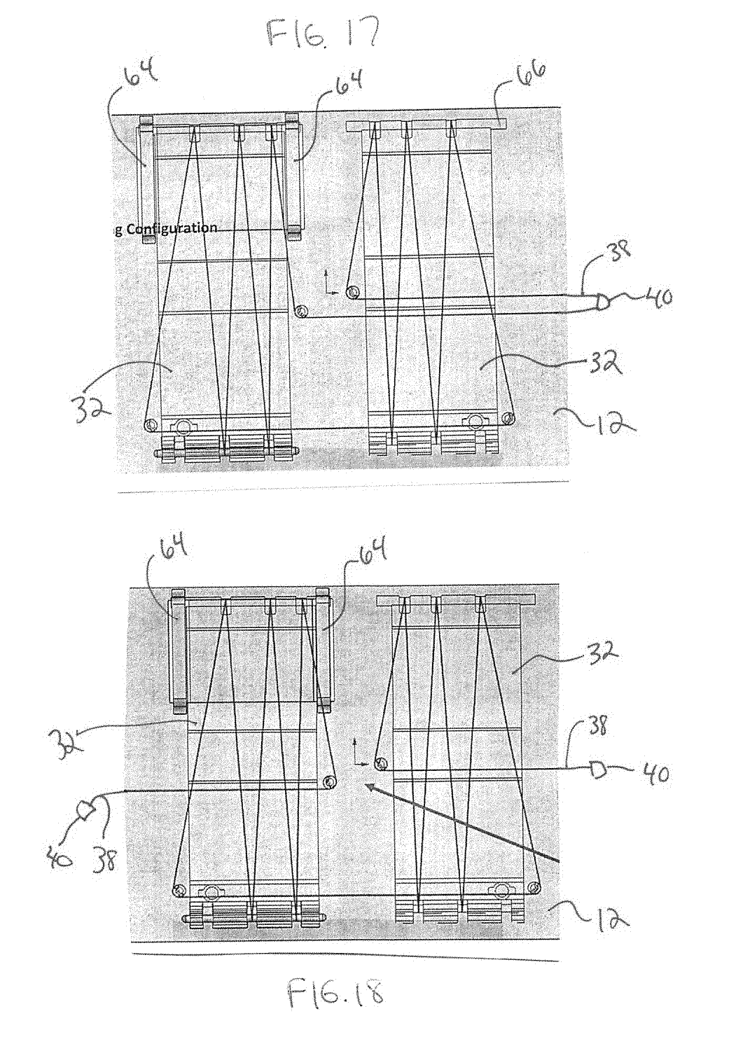

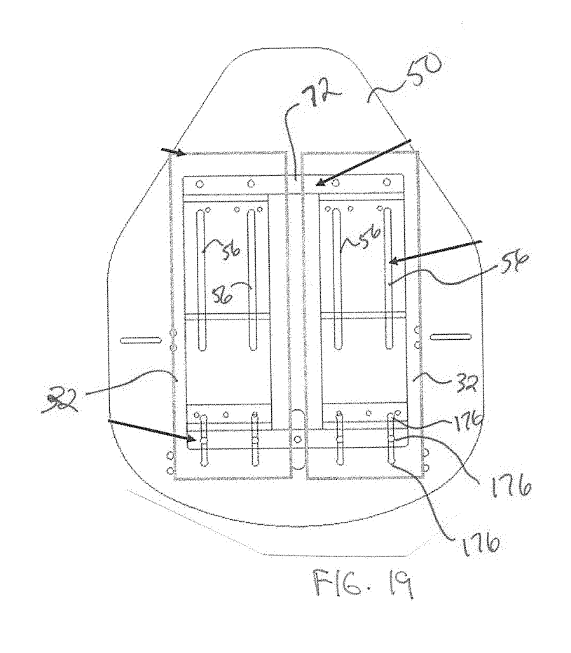

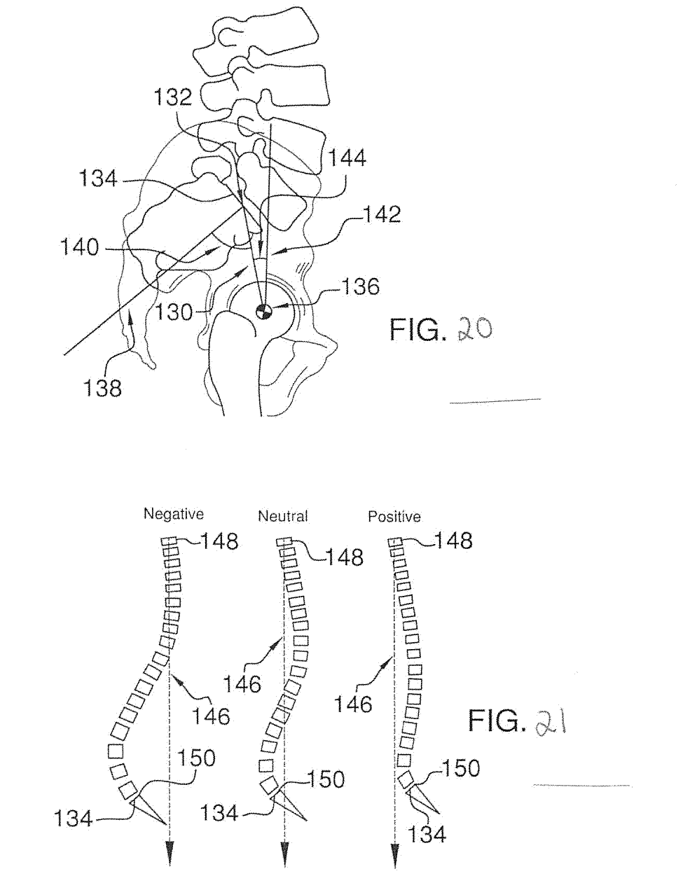

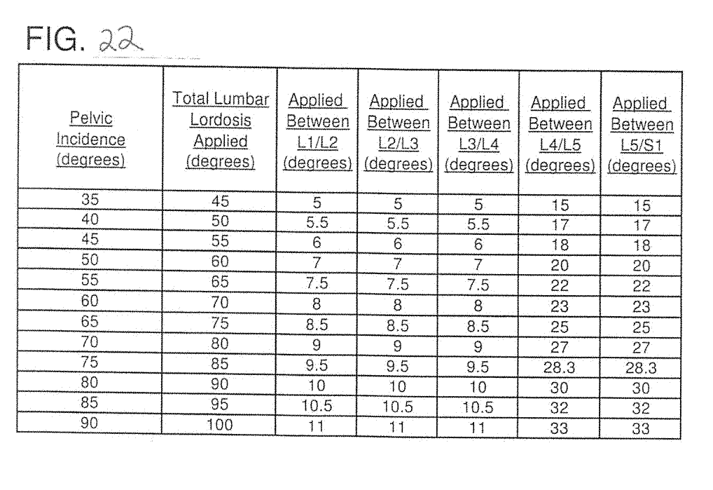

With reference now to the drawings, and in particular to FIGS. 1 through 24 thereof, a new back brace device embodying the principles and concepts of an embodiment of the disclosure and generally designated by the reference numeral 10 will be described.

As best illustrated in FIGS. 1 through 24, the lordosis adjustable back brace device 10 generally comprises a belt 12 having a pair of ends 14,16 and a pair of elongated lateral edges 18,20. The belt 12 is configured for extending around a torso 22 of a human. The belt 12 has a front closure 24 wherein the belt 12 is positionable in a closed loop 26 having an inner surface 28 and an outer surface 30. A support panel 32 is elongated and coupled to the belt 12 in a perpendicular orientation relative to the length of the belt 12 such that the support panel 32 projects from the inner surface 28 of the closed loop 26 towards a center of the closed loop 26 wherein the support panel 32 is configured for abutting the torso 22. The support panel 32 has a curved surface 34 facing the center of the closed loop 26. The front closure 24 may be of a conventional type utilizing a buckle, overlapping hook and loop fastener sections, a strap extending through a loop or loops, or extension of the end 14 through a loop 36 allowing folding over of the end 14 and securement of the end 14 to the belt 12 using hook and loop fastener or the like. An example of a front closure may be found in the closure of issued U.S. Pat. No. 8,142,377.

A circumference of the closed loop 26 may be varied by adjustment of the front closure 24 or the circumference of the closed loop 26 may be set through adjustment of one of more other conventional adjustment mechanisms such that the front closure does not adjust to change the circumference of the closed loop 26. In the latter situation, the belt 12 may be provided in a length corresponding generally to torso size and fitted by adjustment of the other adjustment mechanisms which may then be set to prevent unwanted variation in the length of the belt 12 after fitting. It is contemplated that the front closure 24 may still provide minor adjustment to the circumference if desired after fitting while still allowing the fitting to establish desired positioning of the elements of the device 10 on the torso 22.

A curvature of the curved surface 34 extends between the lateral edges 18,20 of the support panel 32 is adjustable. The curvature may correspond to a desired formula or pattern such as a formula for correction of lumbar lordosis dependent on pelvic incidence. This exemplary formula and correction of lumbar lordosis is described in issued U.S. Pat. No. 9,237,956. The curvature of the curved surface 34 of the support panel 32 is adjustable such that the curvature is configured for adjustment corresponding to lumbar lordosis between 0 and 90 degrees of curvature. Adjustment may be achieved through use of an adjustment line 38 engaging the support panel 32 such that pulling on the adjustment line 38 adjusts the curvature of the curved surface 34 of the support panel 32. The adjustment line 38 may be linked to an array of pulleys, loops, channels, or the like to provide mechanical advantage in providing an adjustment force to the support panel 32. Use of such mechanisms is conventionally known in braces such as found in issued U.S. Pat. No. 6,602,214 disclosing specifically a pulley like system for laterally tightening a brace using a single pull line. A free end 40 of the adjustment line 38 is engageable to the belt 12 wherein the free end 40 of the adjustment line 38 is coupled to the belt 12. The free end 40 of the adjustment line 38 is coupled to the belt 12 in a static position wherein tension in the adjustment line 38 maintains the curvature of the curved surface 34 of the support panel 32. Each of a plurality of marks 42 is positioned on the outer surface 30 of the closed loop 26. The marks 42 are spaced along a length of the belt 12 wherein positioning of the free end 42, which for purposes of this application may effectively be the position of a fastener used to secure the adjustment line 38 to the belt 12, adjacent or corresponding to a selected one of the marks 42 corresponds to a degree of adjustment of the curvature of the curved surface 34. Thus, once fitted, the curvature of the curved surface is consistently adjustable to a desired degree of lordosis by securement of the free end 40 to corresponding to the appropriate corresponding mark 42.

Control of the adjustment of the curvature of the curved surface 24 of the support panel 32 may be achieved in various ways including but not limited to the following described mechanisms. In an embodiment of the invention, each of a plurality of bend lines 44 extends laterally across the support panel 32. The bend lines 44 are spaced apart between an upper end 46 and a lower end 48 of the support panel 32 wherein adjustment of a distance between the upper end 46 and the lower end 48 of the support panel 32 provided by pulling of the adjustment line 38 results in bending of the support panel 32 at the bend lines 44. Thus, the positioning of the bend lines 44 determines alteration of the curvature of the curved surface 34 of the support panel 32.

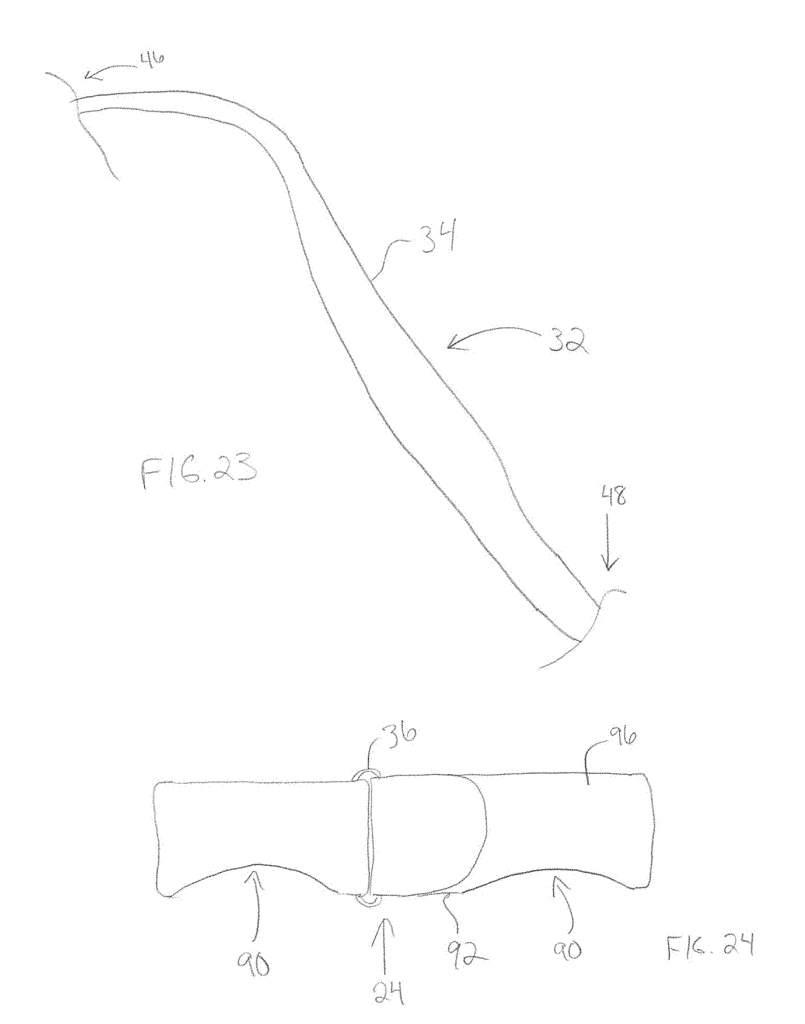

Alternatively as shown in FIG. 23, or in combination with one or more bend lines, a thickness of the support panel 32 extending laterally across the support panel 32 is varied between the upper end 46 and the lower end 48 of the support panel 32. The variation in thickness contemplates a distinction from distinct bend lines 44 as described above as the variation in thickness may be smoothly transitioned along the length of the support panel 32 between the upper end 46 and the lower end 48. However, the variation in thickness may still determine alteration of the curvature of the curved surface 34 of the support panel 32.

The support panel 32 may be coupled directly to the belt 12. Alternatively, the support panel 32 may be coupled to a backing panel 50 which is removably couplable to the belt 12 in a conventional manner using fasteners such as snaps, hook and loop fasteners, or the like. The backing panel 50 is typically stiff and secured rigidly relative to the belt providing additional bracing to a back of the user. The backing panel 50 has an uppermost edge 52 and a bottommost edge 54. A length between the bottommost edge 54 and the topmost edge 52 can range between 7.0 and 40.0 inches. The backing panel 50 may be one of a plurality of backing panels 50 having varied lengths within the above range such that support of the back of the user is customizable to cover a desired portion of the user's back. For a small brace the range may be between 7.0 and 8.5 inches, an LSO brace may be up to 14.5 inches, and a TLSO brace may be up to 40.0 inches. The support panel 32 is coupled to the backing panel 50 closer to the bottommost edge 54 than the topmost edge 52 and the curved surface 34 of the support panel 32 has a length and is positioned on the belt 12 or backing panel 50 such that the support panel 32 is configured for positional alignment extending between a L1 vertebral body and a S1 vertebral body. Thus, the device 10 is designed to be adjustable to provide lordosis support complementary to a desired lordosis for the back such as is exemplified in U.S. Pat. No. 9,237,956.

Adjustment of the curvature of the curved surface 34 is generally achieved through application of tension to draw the upper end 46 of the support panel 32 and the lower end 48 of the support panel 32 together. This may be achieved in a variety of ways including the lower end 48 being coupled to the belt 12, directly or through use of the backing panel 50, in a fixed position. However, the lower end 48 may be pivotal at the fixed position to facilitate formation of the desired curvature. The upper end 46 of the support panel 32 is slidably coupled to the belt 12, again either directly or through use of the backing panel 50. The curvature of the curved surface 34 of the support panel 32 is adjusted by sliding of the upper end 46 of the support panel 32 relative to the belt 12. Each of a plurality of slots 56 may extend through the support panel 32. Each of the slots 56 is parallel to a length of the support panel 32. Generally, each of a plurality of fasteners 58 is coupled to the belt 12, either directly or by virtue of being coupled directly to the backing panel 50. Each of the fasteners 58 extends through an associated one of the slots 56 wherein the fastener 58 is slidable within the associated one of the slots 56. Thus, manipulation of the adjustment line 38 translates to linear application of tension in the support panel 32 to drive adjustment of the curvature of the curved surface 34 of the support panel 32. The plurality of slots 56 may be an upper pair 60 of slots 56 positioned adjacent to the upper end 46 of the support panel 32, a lower pair 62 of slots 56 positioned adjacent to the lower end 48 of the support panel 32, or a combination of the upper pair 60 and lower pair 62 may be simultaneously employed with or without additional structure to fix the position of the support panel 32 relative to the belt 12 or backing panel 50 depending on whether it is desired for the support panel 32 as a whole to have a set curvature but be able to move relative to the belt 12 or backing panel 50.

Alternatively, as shown in FIG. 11, sliding and adjustment of the support panel 32 may be achieved through the use of brackets 64 and lateral extensions 66. The brackets 64 are coupled to the belt 12 or backing panel 50. The brackets 64 are positioned laterally spaced from the support panel 32 adjacent to either the upper end 46, lower end 48, or both. Each of a plurality of lateral extensions 66 is coupled to the support panel 32. Each lateral extension 66 engages and slides within a respective track 68 formed by an associated one of the brackets 64. The lateral extensions 66 may be integral with a bar 74 to which the upper end 46 is pivotally coupled to again facilitate desired shaping of the curvature as tension is adjusted.

In another embodiment as shown in FIG. 24, a belt 96 is similarly constructed to belt 12 but for a pair of indents 90 extending into a lower edge 92 of the belt 12. The front closure 24 is positioned between the indents 90 wherein each of the indents 90 is positioned to be aligned with human legs extending from the torso wherein the indents 90 each receive a respective one of the human legs. Thus, the belt 96 is configured to remain in a static position on the human torso when each of the human legs is pivoted forward at a respective hip joint.

The support panel 32 may also be provided with a curved medial portion 76 and end portions 78 extending from the curved medial portion 76 to facilitate coupling to the belt 12 or backing panel 50. In such an embodiment, the distance between a top edge 80 and bottom edge 82 of the curved medial portion 76 provides the effective curvature to be adjusted. The curved medial portion 76 may be further defined between pivotal connections 84 facilitating adjustment of the curvature while the end portions 78 remain adjacent to the belt 12.

FIGS. 14 and 16 show a plurality of channels 170 occupied by a bar 172 through which the adjustment line 38 may extend. FIG. 19 shows a plurality of notches 176 which may be used in the various embodiments as points where the support panel 32 may be fixed to the belt 12 or backing panel 50. Thus, the notches 176 provide some initial gross setting and adjustment possibilities which may then be fixed such that tension in the adjustment line 38 provides fine adjustment to the curvature of the curved surface 34 of the support panel 32.

The device 10 may be provided with a pair of support panels 32. Each of the support panels 32 is coupled to the belt 12 such that the support panel 32 projects from the inner surface 28 of the closed loop 26 towards a center of the closed loop 26 wherein each support panel 32 is configured for abutting the torso. The pair of support panels 32 is laterally aligned and spaced apart such that the curvature of the curved surfaces 34 of the support panels 32 defines a gap 70 between the support panels 32. Thus, the pair of support panels 32 is configured for spacing the belt 12 from direct contact with the user along the spine by aligning the gap 70 with the spine while the belt 12 is secured around the torso 22. An alignment bar 72 may be coupled to and extend between the pair of support panels 32 inhibiting independent movement of the support panels 32 and therefore promoting similar curvature in the curved surfaces 34 of the support panels 32. Further, a single adjustment line 74 engaging both support panels 32 may be utilized for a two support panel configuration. Pulling on the single adjustment line 74 shortens the distance between the upper end 46 and the lower end 48 of each of the support panels 32.

Alternatively, each of a pair of adjustment lines 76 is engaged to a respective one of the support panels 32 wherein a distance between the upper end 46 and the lower end 48 of each of the support panels 32 is separately adjustable by manipulation of the adjustment lines 76. Similar to the above description, each support panel 32 may be coupled directly to the belt 12 or to the backing panel 50.

The specific shape of the curvature of the support panel may correspond to a desired or preferred formula to be complementary to desired shape of the spine. By way of example, a cumulative angle between each of multiple pairs of vertebral bodies may be supported relative to pre-determined reference lines determined from measurements of pelvic incidence. Alternatively, other body references may be utilized to determine alternative formulations to achieve or determine the desired shape for the curvature of the curved surface 34. Additional factors may include pelvic tilt and C7 plumb line. Desired angles between each pair of vertebral bodies may be determined according to the chart of FIG. 21. Additional factors for each particular patient may also be taken into account in addition to pelvic incidence, pelvic tilt and C7 plumb line when determining exact lordosis corrections between specific adjacent vertebral bodies. The shape of the support panel may then be designed to correspond to be complementary to the exact lordosis corrections.

As shown in FIG. 22, total lumbar lordosis (LL) required is well estimated by the formula LL=pelvic incidence (PI) plus ten (10) degrees, or using a more rigid formula wherein LL=0.56×PI+33. Current practice is for two-thirds of the cumulative lordosis correction to be provided in between the L4/5 and L5/S1 vertebral bodies and the remaining correction divided evenly between L1/2, L2/3, and L3/4 vertebral bodies. These formulas provide a framework for determining the patient's sagittal imbalance that may need correction. Other factors may be included in determining total corrective lordosis if there is a positive sagittal imbalance on standing 36-inch radiographs as determined by the C7 plumb line. For example, a patient with hyper thoracic kyphosis may require additional lumbar lordosis to provide desired results.

As demonstrated in FIG. 20, pelvic incidence is measured by drawing a line 130 from the midpoint 132 of the S1 superior endplate 134 to the center of the midpoint 136 between both femoral heads. A second line 138 is drawn parallel to a perpendicular line from the S1 endplate 134 with the starting point at the midpoint 132 of the S1 superior endplate 134. The angle 140 measured between these two lines 130,138 is the pelvic incidence. This measurement defines the relationship between the spine and the pelvis and can be used to predict the amount of lumbar lordosis needed to maintain a neutral sagittal balance. As also shown in FIG. 20, pelvic tilt is measured using the line 130 from the midpoint 136 between the center of the femoral heads to the midpoint 132 of the S1 superior endplate 134. A vertical reference line 142 is drawn vertically from the midpoint 136 between both femoral heads. An angle 144 created between the line 130 and the vertical reference line 142 represents the pelvic tilt. The angle 144 helps the surgeon determine if the patient is compensating for lack of lumbar lordosis by increasing their pelvic tilt to maintain a neutral sagittal balance. As shown in FIG. 21, C7 plumb line 146 is drawn on a 36 inch lateral radiograph by finding the middle of the C7 vertebral body 148 and drawing a vertical line downward. A measurement from the posterior superior corner 150 of the S1 vertebral endplate 134 to the C7 plumb line 146 is measured. This determines the patient's sagittal balance. A positioning of the C7 plumb line 146 posterior to the superior corner 154 of the S1 endplate is considered a negative sagittal balance, and a positive value is noted when the C7 plumb line 146 is anterior to the posterior superior corner 150 of the S1 vertebral endplate 134. A positive sagittal balance value of greater than 5 centimeters is considered abnormal.

With the above information for a particular patient, incremental adjustment between adjacent vertebral bodies, and the corresponding complementary shape desired for the support panel 32 may be determined consistent with FIG. 22 and any additional factors deemed necessary to provide the desired total cumulative support of the spine relative to the position of the pelvis to support sagittal balance.

In an embodiment of the invention, the curvature may be adjusted by utilizing flexible cords connecting rear surfaces of a supporting panel relative to one or more fixed points relative to a belt. Tension in the flexible cord produces a curvature in the surface of the supporting panel between the belt and the fixed points. This may again be provided for with one or more supporting panels. This can be structured to result in the desired curvature to the surface abutting the patient's back and application of the desired curvature to the spine of the patient. In this embodiment, two cords may be used, one for each lateral side of the device, but these cords may also be interconnected or adjusted through a single mechanism for providing consistent or equivalent force to each cord such that the adjustments to each side of the front section are consistent.

Additional embodiments may provide for adjustment of the curvature of the surface of the front section in different manners including but not limited to use of air pockets inflated or deflated as needed or adjustable length braces or rods extending between the main section and the front section.

In use, the support panel 32 of support panels 32 are shaped with the bend lines 44 or changes in thickness to provide the desired adjustment to the curvature as the adjustment line 38 is pulled. The support panel 32 is coupled to the belt 12 to rest against the back of the user in the desired position. The belt 12 is secured to the torso and the adjustment line 38 is pulled to adjust the curvature of the curved surface 34 either to a comfortable position, or using marks 42 to a specific setting to be complementary to a prescribed degree of lordosis for the particular user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A back support brace device comprising:
a belt having a pair of ends and a pair of elongated lateral edges wherein said belt is configured for extending around a torso of a human, said belt having a front closure wherein said belt is positionable in a closed loop having an inner surface and an outer surface; and
a support panel coupled to said belt such that said support panel projects from said inner surface of said closed loop towards a center of said closed loop wherein said support panel is configured for abutting the torso, said support panel having a curved surface facing said center of said closed loop, a curvature of said curved surface extending between said lateral edges of said support panel being adjustable.

2. The device of claim 1, further comprising said curvature of said curved surface of said support panel being adjustable such that said curvature is configured for adjustment corresponding to lumbar lordosis between 0 and 90 degrees of curvature.

3. The device of claim 1, further comprising an adjustment line engaging said support panel such that pulling on said adjustment line adjusts said curvature of said curved surface of said support panel.

4. The device of claim 3, further comprising a free end of said adjustment line being engageable to said belt wherein said free end of said adjustment line is coupled to said belt.

5. The device of claim 3, further comprising said free end of said adjustment line being coupled to said belt in a static position wherein tension in said line maintains said curvature of said curved surface of said support panel.

6. The device of claim 4, further comprising a plurality of marks being positioned on said outer surface of said closed loop, said marks being spaced along a length of said belt wherein positioning of said free end of said belt adjacent to a selected one of said marks corresponds to a degree of adjustment of said curvature of said curved surface.

7. The device of claim 1, further comprising a plurality of bend lines extending laterally across said support panel, said bend lines being spaced apart between an upper end and a lower end of said support panel wherein adjustment of a distance between said upper end and said lower end of said support panel determines alteration of said curvature of said curved surface of said support panel.

8. The device of claim 1, further comprising a thickness of said support panel extending laterally across said support panel being varied between an upper end and a lower end of said support panel wherein adjustment of a distance between said upper end and said lower end determines alteration of said curvature of said curved surface of said support panel.

9. The device of claim 1, further comprising said support panel being coupled directly to said belt.

10. The device of claim 1, further comprising said support panel being coupled to a backing panel, said backing panel being removably couplable to said belt.

11. The device of claim 10, further comprising said backing panel having an uppermost edge and a bottommost edge, said support panel being coupled to said backing panel closer to said bottommost edge than said topmost edge.

12. The device of claim 11, further comprising a length between said bottommost edge and said topmost edge being between 7.0 and 40.0 inches.

13. The device of claim 12, further comprising said curved surface of said support panel having a length and being positioned on said backing panel such that said support panel is configured for positional alignment extending between a L1 vertebral body and a S1 vertebral body.

14. The device of claim 1, further comprising a bottom end of said support panel being coupled to said belt in a fixed position, an upper end of said support panel being slidably coupled to said belt, said curvature of said curved surface of said belt being adjusted by sliding of said upper end of said support panel relative to said belt.

15. The device of claim 1, further comprising:
a plurality of slots extending through said support panel, each of said slots being parallel to a length of said support panel; and
a plurality of fasteners, each said fastener being coupled to said belt, each of said fasteners extending through an associated one of said slots wherein said fastener is slidable within said associated one of said slots.

16. The device of claim 15, further comprising said plurality of slots being an upper pair of slots positioned adjacent to an upper end of said support panel.

17. The device of claim 15, further comprising said plurality of slots being a lower pair of slots positioned adjacent to a lower end of said support panel.

18. The device of claim 15, further comprising said plurality of slots including an upper pair of slots positioned adjacent to an upper end of said support panel and a lower pair of slots positioned adjacent to a lower end of said support panel.

19. A back support brace comprising:
a belt having a pair of ends and a pair of elongated lateral edges wherein said belt is configured for extending around a torso of a human, said belt having a front closure wherein said belt is positionable in a closed loop having an inner surface and an outer surface; and
a pair of support panels, each said support panel being coupled to said belt such that said support panel projects from said inner surface of said closed loop towards a center of said closed loop wherein each said support panel is configured for abutting the torso, each said support panel having a respective curved surface facing said center of said closed loop, a curvature of each said curved surface extending between said lateral edges of said support panel being adjustable.

20. The device of claim 19, further comprising said curvature of said curved surface of each said support panel being adjustable such that said curvature of each said curved surface is configured for adjustment corresponding to lumbar lordosis between 0 and 90 degrees of curvature.

21. The device of claim 19, further comprising said pair of support panels being laterally aligned and spaced apart such that said curvature of said curved surfaces of said support panels defines a gap between said support panels wherein said support panels are configured for spacing said belt from a human spine while said belt is secured around the torso.

22. The device of claim 21, further comprising an alignment bar coupled to and extending between said pair of support panels wherein said alignment bar inhibits independent movement of said support panels.

23. The device of claim 19, further comprising a single adjustment line engaging both support panels wherein pulling on said single adjustment line shortens said distance between said upper end and said lower end of each of said support panels.

24. The device of claim 19, further comprising a pair of adjustment lines, each adjustment line being engaged to a respective one of said support panels wherein a distance between said upper end and said lower end of each of said support panels is separately adjustable by manipulation of said adjustment lines.

25. The device of claim 19, further comprising each said support panel being coupled directly to said belt.

26. The device of claim 19, further comprising each said support panel being coupled to a backing panel, said backing panel being removably couplable to said belt.

27. The device of claim 26, further comprising said backing panel having an uppermost edge and a bottommost edge, each said support panel being coupled to said backing panel closer to said bottommost edge than said topmost edge.

28. The device of claim 27, further comprising a length between said bottommost edge and said topmost edge being between 7.0 and 40.0 inches.

29. The device of claim 28, further comprising said curved surface of each said support panel having a length and being positioned on said backing panel such that each said support panel is configured for positional alignment extending laterally adjacent to a L1 vertebral body and a S1 vertebral body.

30. A back support brace comprising:
a belt having a front closure, said belt being elongated wherein said belt is configured for extending around a torso of a human; and
a pair of support panels, each said support panel being coupled to said belt, each said support panel being elongated, each said support panel being oriented perpendicular to said belt, said support panels being spaced apart wherein said support panels are configured for positioning on opposite sides of a spine, a distance between an upper end and a lower end of each said support panel being adjustable wherein a curvature of a curved surface of each said support panel between said upper end and said lower end and configured for abutting the torso is adjustable.

31. The device of claim 30, further comprising said curvature of said curved surface of each said support panel being adjustable such that said curvature is configured for adjustment corresponding to lumbar lordosis between 0 and 90 degrees of curvature.

32. The device of claim 30, further comprising said pair of support panels being laterally aligned and spaced apart such that said curvature of said curved surfaces of said support panels defines a gap between said support panels and said belt wherein said support panels are configured for spacing said belt from the human spine.

33. The device of claim 31, further comprising an alignment bar coupled to and extending between said pair of support panels wherein said alignment bar inhibits independent movement of said support panels.

34. The device of claim 30, further comprising a single adjustment line engaging both support panels wherein pulling on said single adjustment line shortens said distance between said upper end and said lower end of each of said support panels.

35. The device of claim 30, further comprising a pair of adjustment lines, each adjustment line being engaged to a respective one of said support panels wherein a distance between said upper end and said lower end of each of said support panels is separately adjustable by manipulation of said adjustment lines.

36. The device of claim 30, further comprising each said support panel being coupled directly to said belt.

37. The device of claim 30, further comprising each said support panel being coupled to a backing panel, said backing panel being removably couplable to said belt.

38. The device of claim 37, further comprising said backing panel having an uppermost edge and a bottommost edge, each said support panel being coupled to said backing panel closer to said bottommost edge than said topmost edge.

39. The device of claim 38, further comprising a length between said bottommost edge and said topmost edge being between 7.0 and 40.0 inches.

40. The device of claim 39, further comprising said curved surface of each said support panel having a length and being positioned on said backing panel such that said support panel is configured for positional alignment extending laterally adjacent to a L1 vertebral body and a S1 vertebral body.

41. A back support brace device comprising:
a belt having a front closure, said belt being elongated wherein said belt is configured for extending around a torso of a human; and
a support panel coupled to said belt, said support panel being elongated, said support panel being oriented perpendicular to said belt, a distance between an upper end of said support panel and a lower end of said support panel being adjustable wherein a curvature of a curved surface of said support panel relative to said belt is adjustable.

42. The device of claim 41, further comprising said curvature of said curved surface of said support panel being adjustable such that said curvature is configured for adjustment corresponding to lumbar lordosis between 0 and 90 degrees of curvature.

43. The device of claim 41, further comprising an adjustment line engaging said support panel such that pulling on said adjustment line shortens said distance between said upper end and said lower end of said support panel.

44. The device of claim 43, further comprising a free end of said adjustment line being engageable to said belt wherein said free end of said adjustment line is coupled to said belt.

45. The device of claim 43, further comprising said free end of said adjustment line being coupled to said belt in a static position wherein tension in said line maintains said curvature of said curved surface of said support panel.

46. The device of claim 45, further comprising a plurality of marks being positioned on an exterior face of said belt, said marks being spaced along a length of said belt wherein positioning of said free end of said belt adjacent to a selected one of said marks corresponds to a degree of adjustment of said curvature of said curved surface.

47. The device of claim 41, further comprising a plurality of bend lines extending laterally across said support panel wherein said curved surface of said support panel is determined by relative positioning of said bend lines.

48. The device of claim 41, further comprising a thickness of said support panel extending laterally across said support panel being varied between said upper end and said lower end of said support panel wherein adjustment of said distance between said upper end and said lower end determines alteration of said curvature of said curved surface of said support panel.

49. The device of claim 41, further comprising said support panel being coupled directly to said belt.

50. The device of claim 41, further comprising said support panel being coupled to a backing panel, said backing panel being removably couplable to said belt.

51. The device of claim 50, further comprising said backing panel having an uppermost edge and a bottommost edge, said support panel being coupled to said backing panel closer to said bottommost edge than said topmost edge.

52. The device of claim 51, further comprising a length between said bottommost edge and said topmost edge being between 7.0 and 40.0 inches.

53. The device of claim 52, further comprising said curved surface of said support panel having a length and being positioned on said backing panel such that said support panel is configured for positional alignment extending between a L1 vertebral body and a S1 vertebral body.

54. The device of claim 41, further comprising said bottom end of said support being coupled to said belt in a fixed position, said upper end of said support panel being slidably coupled to said belt.

55. The device of claim 41, further comprising:
a plurality of slots extending through said support panel, each of said slots being parallel to a length of said support panel; and
a plurality of fasteners, each said fastener being coupled to said belt, each of said fasteners extending through an associated one of said slots wherein said fastener is slidable within said associated one of said slots.

56. The device of claim 55, further comprising said plurality of slots being an upper pair of slots positioned adjacent to said upper end of said support panel.

57. The device of claim 55, further comprising said plurality of slots being a lower pair of slots positioned adjacent to said lower end of said support panel.

58. The device of claim 55, further comprising said plurality of slots including an upper pair of slots positioned adjacent to said upper end of said support panel and a lower pair of slots positioned adjacent to said lower end of said support panel.

59. A back support brace comprising:
a belt, said belt having a lower edge and an upper edge, said belt having a front closure, said belt being elongated wherein said belt is configured for extending around a torso of a human;
a support panel coupled to said belt, an exposed surface of said support panel relative to said belt being configured for abutting a back of the human; and
a pair of indents extending into said lower edge, said front closure being positioned between said indents wherein each of said indents is configured to be aligned with human legs extending from the torso wherein said indents each receive a respective one of the human legs such that said belt is configured to remain in a static position on the human torso when each of the human legs is pivoted forward at a respective hip joint.

60. A back support brace device comprising:
a belt having a front closure, said belt being elongated wherein said belt is configured for extending around a torso of a human; and
a support panel coupled to said belt, said support panel being elongated, said support panel being oriented perpendicular to said belt, said support panel having an exposed surface configured to abut the torso, said exposed surface having a curved medial portion, a distance between a top edge and a bottom edge of said curved medial portion of said exposed surface of said support panel being adjustable wherein a curvature of said curved medial portion of said exposed surface of said support panel is adjustable.

61. The device of claim 60, further comprising an adjustment line engaging said support panel such that pulling on said adjustment line shortens a distance between said upper end and said lower end of said support panel wherein said distance between said upper and lower ends of said support panel is adjustable such that said curvature of said curved medial portion of said exposed surface of said support panel is adjustable.

62. The device of claim 61, further comprising a free end of said adjustment line being engageable to said belt wherein said free end of said adjustment line is coupled to said belt.

63. The device of claim 64, further comprising said free end of said adjustment line being coupled to said belt in a static position wherein tension in said line maintains said curvature of said curved medial portion of said exposed surface of said support panel.

64. The device of claim 62, further comprising a plurality of marks being positioned on an exterior face of said belt, said marks being spaced along a length of said belt wherein positioning of said free end of said belt adjacent to a selected one of said marks corresponds to a degree of adjustment of said curvature of said curved medial portion of said exposed surface.

65. The device of claim 60, further comprising a plurality of bend lines extending laterally across said support panel wherein said curved medial portion of said exposed surface of said support panel bends at said bend lines when said distance between said top edge and said bottom edge of said curved medial portion is adjusted whereby said curvature of said curved medial portion of said exposed surface is determined by relative positioning of said bend lines.

66. The device of claim 60, further comprising a thickness of said support panel extending laterally across said support panel within said curved medial portion of said exposed surface being varied between said top edge and said bottom edge of said curved medial portion wherein adjustment of said distance between said top edge and said bottom edge determines alteration of said curvature of said curved medial portion of said exposed surface of said support panel.

67. The device of claim 60, further comprising said support panel being coupled directly to said belt.

68. The device of claim 60, further comprising said support panel being coupled to a backing panel, said backing panel being removably couplable to said belt.

69. The device of claim 60, further comprising a bottom portion of said support panel extending from said curved medial portion being coupled to said belt in a fixed position, an upper portion of said support panel extending from said curved medial portion being slidably coupled to said belt.

70. The device of claim 60, further comprising:
a plurality of slots extending through said support panel, each of said slots being parallel to a length of said support panel; and
a plurality of fasteners, each said fastener being coupled to said belt, each of said fasteners extending through an associated one of said slots wherein said fastener is slidable within said associated one of said slots.

71. The device of claim 70, further comprising said plurality of slots being an upper pair of slots positioned within said upper portion of said support panel.

72. The device of claim 70, further comprising said plurality of slots being a lower pair of slots positioned within said lower portion of said support panel.

73. The device of claim 70, further comprising said plurality of slots including an upper pair of slots positioned within said upper portion of said support panel and a lower pair of slots positioned within said lower portion of said support panel.
